(19) United States
(12) Patent Application Publication
(10) Pub. No.: US 2022/0403553 A1
(43) Pub. Date: Dec. 22, 2022

(54) METHOD FOR SCREENING LIBRARIES

(71) Applicant: GLAXOSMITHKLINE INTELLECTUAL PROPERTY DEVELOPMENT LIMITED, Brentford (GB)

(72) Inventors: Brent DORR, Collegeville, PA (US); Genaro SCAVELLO, Collegeville, PA (US)

(21) Appl. No.: 17/640,630

(22) PCT Filed: Sep. 10, 2020
(86) PCT No.: PCT/EP2020/075355

§ 371 Date: Mar. 4, 2022

Related U.S. Application Data
(60) Provisional application No. 62/899,352, filed on Sep. 12, 2019.

(51) Int. Cl. C40B 40/06; C40B 50/06; C12N 15/10; C12Q 1/686

(52) U.S. Cl. CPC C40B 40/06; C40B 50/06; C12N 15/1093; C12Q 1/686

(57) ABSTRACT

The present invention relates to a method for identifying a DNA target sequence of an endonuclease. Substrate libraries for use in this method and methods of engineering endonucleases to have improved cleavage efficiency for a particular substrate form other aspects of the invention.

### FIELD OF THE INVENTION

The present invention relates to a method for identifying a DNA target sequence of an endonuclease. Substrate libraries for use in this method and methods of engineering endonucleases to have improved cleavage efficiency for a particular substrate form other aspects of the invention.

### BACKGROUND OF THE INVENTION

Endonucleases capable of cleaving a single site within the genome have enormous potential for genome editing by stimulating either non homologous end joining or homologous recombination and a variety of engineered endonucleases have entered clinical trials, including CCR5-2246 (targeting human CCR5) and VR24684 (targeting human VEGF-A promoter). Despite the ability to engineer certain nucleases (e.g. RNA guided nucleases such as Cas9/guide RNA, TALENs or Zinc Finger Nucleases) to have specificity for a particular unique target sequence, it has become apparent that these nucleases may also interact with and cleave at other off-target sequences. Indeed some engineered nucleases have been linked to cellular toxicity and oncogenicity. The FDA noted that specificity of cleavage would need to be considered when approving therapeutic endonucleases in a Science Board Meeting on 15 Nov. 2016.

In view of the concerns around off-target cleavage, various attempts have been made to characterise the cleavage of endonucleases on target sites closely related to those for which endonucleases were engineered to cleave. WO2018/119010 describes a method in which an oligonucleotide library is used that is simple to produce at scale and the method is compatible with automation. However, the method suffers from a low signal to noise ratio and high cleavage rates are required in order to detect a signal. As a result, this method is conducted using non-physiological enzyme:DNA stoichiometry which could itself lead to artefacts.

A method capable of overcoming the disadvantages associated with the prior art is required.

### SUMMARY OF THE INVENTION

In a first aspect, the present invention provides a substrate library, comprising a plurality of DNA substrates, wherein each substrate within the library contains a putative target sequence that is 5′ of an identifier DNA sequence capable of uniquely identifying said putative target sequence, which is 5′ of a sequence that is identical to a reverse PCR primer, and wherein the double stranded DNA substrates within the library differ from one another only by the putative target sequences and identifier DNA sequences.

In one embodiment, the substrate library comprises a plurality of double stranded DNA substrates.

In a further aspect, the invention provides a method for preparing a substrate library comprising a plurality of double stranded DNA substrates as defined herein. The method comprises a step of PCR amplification of a plurality of putative target sequences flanked with a) a sequence complementary to a library forward primer and b) a sequence identical to a portion of a library reverse primer sequence, with said library forward primer and library reverse primer, wherein the library reverse primer is a heterogeneous mixture of DNA sequences containing distinct identifier sequences located 5′ of a sequence common to all sequences that is complementary to the reverse primer, and wherein the number of distinct identifier sequences is in molar excess of the number of putative target sequences.

In another aspect, the invention provides a method for identifying a DNA target sequence of an endonuclease, comprising the following steps:
a) contacting a substrate library as described herein with an endonuclease under suitable conditions to permit cleavage;
b) ligating the endonuclease treated library with a DNA sequence including a sequence complementary to a “cleavage” PCR primer;
c) PCR amplification of the cleaved substrate with cleavage and reverse PCR primers; and
d) sequencing of the amplified PCR product;

wherein a DNA target sequence in the cleaved product is identified via the sequence of the identifier sequence.

In yet another aspect, the invention provides a method for engineering endonucleases, comprising:

a) conducting the method for identifying a DNA target sequence of an endonuclease as described herein with a first endonuclease and at least two other endonucleases that differ from the first endonuclease by a single amino acid change at different positions within the endonuclease amino acid sequence using the same substrate library;

b) comparing the efficiency of cleavage of each endonuclease tested in step a) at a particular substrate;

c) identifying at least two amino acid changes at different positions that improve the efficiency of cleavage;

d) producing a variant endonuclease containing the at least two amino acid changes identified in step c).

In yet another aspect, the invention provides variant endonucleases obtained by the methods described herein.

### BRIEF DESCRIPTION OF THE DRAWINGS

FIG. 1 shows a schematic representation of steps a) to c) of the method for identifying a DNA target sequence of an endonuclease. FIG. 1A shows a double stranded substrate library, including the putative target sequence, identifier sequence and the sequence complementary to the reverse primer. FIG. 1B shows the cleaved substrate library. In this representation, putative target sequence 2 is the only putative target sequence to have been cleaved. FIG. 1C shows the library following ligation to a cleavage primer. FIG. 1D shows the amplified PCR product resulting from PCR amplification using the cleavage and reverse primers.

FIG. 2 shows the frequency of the individual DNA substrates in the substrate library characterised in Example 3. The y axis shows the individual oligonucleotide count frequency.

FIG. 3A shows a 4% agarose gel of the uncut PCR reaction using both wild type Cas9-RNP and R691A mutant Cas9-RNP. FIG. 3B shows a 4% agarose gel of the cut PCR reaction using both wild type Cas9-RNP and R691A mutant Cas9-RNP.

FIG. 4 shows the relative abundance of various numbers of mismatches in the resulting probe library and the relative proportion of unsampled sequences with N=4, 5 or 6 mismatches in the PAM or gRNA regions of the DNA

FIGS. 5 and 6 show the BEESEM-derived binding profiles (A and B) and a comparison of Hifi Cas9 vs wt Cas9 at 1:1 and 1:5 DNA:RNP ratios (C).

FIG. 7 shows a reproducibility trial demonstrating the high reproducibility of two assay runs on the same conditions, and a comparison of the relatively high correlation between each oligo in the pool, compared across one another.

FIG. 8 shows the frequency of the individual DNA substrates in the substrate library characterised in Example 6. The y axis shows the individual oligonucleotide count frequency.

FIG. 9 shows the relative abundance of various numbers of mismatches in the probe library for I-SceI (A) and the relative proportion of unsampled sequences with N=4, 5 or 6 mismatches in the target region of the library (B).

FIG. 10 show the BEESEM-derived binding profile for I-SceI at 50 units/ug of DNA library (A), at 5 units/ug of DNA library (B) and at 0.5 units/ug of DNA library (C)

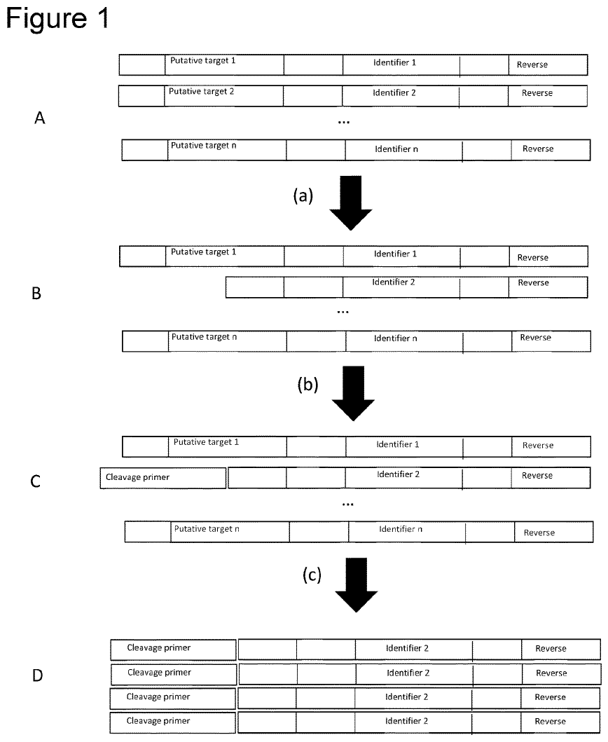

### DETAILED DESCRIPTION OF THE INVENTION

As outlined above, the present invention provides a substrate library, comprising a plurality of DNA substrates, wherein each substrate within the library contains a putative target sequence that is 5′ of an identifier sequence capable of uniquely identifying said putative target sequence, which is 5′ of a sequence that is identical to a reverse PCR primer, and wherein the double stranded DNA substrates within the library differ from one another only by the putative target sequences and identifier DNA sequences.

In the context of this invention, a putative target sequence is a DNA sequence that could potentially be subject to cleavage by an endonuclease. Where the DNA substrate is double stranded, a putative target sequence is a DNA sequence that could potentially be subject to double stranded cleavage by an endonuclease. Where the DNA substrate is single stranded, a putative target sequence is a DNA sequence that could potentially be subject to single stranded cleavage by an endonuclease. The DNA substrates in the substrate library contain distinct putative target sequences with each putative target sequence differing from every other putative target sequence in the library at one or more positions (nucleotides). In one embodiment, all the putative target sequences are the same length.

In one embodiment, the putative target sequence in the library is between 9 and 50 nucleotides in length. In a more particular embodiment, the putative target sequence is between 9 and 40, between 12 and 40, between 12 and 30 nucleotides, between 12 and 25 nucleotides, and between 12 and 20 nucleotides in length. In one embodiment where the endonuclease is a Cas9 nuclease, the putative target sequence is between 18 and 22 nucleotides in length. In another embodiment where the endonuclease is a TALEN, the putative target sequence is between 14 and 20 (monomer) or 32 to 48 (dimer) nucleotides in length. In another embodiment where the endonuclease is a Zinc Finger Nuclease, the putative target sequence is 9 or 15 (monomer) or 22 to 38 (dimer) nucleotides in length. In an embodiment were the endonuclease is a meganuclease, the putative target sequence is between 17 and 24 nucleotides in length.

In one embodiment, putative target sequences are generated randomly. In another embodiment, these are based on knowledge from the literature about a known target sequence of the relevant endonuclease. In this situation, the substrate library contains putative target sequences including the known target sequence and variants of this known target sequence. The variant sequences typically include variants that differ from the target sequence at a single position. In one embodiment, the putative target sequences included comprise the known target sequence for the endonuclease and all possible single variants (all possible single variants refers to the situation where each nucleotide in the sequence is changed to each of the other 3 possible nucleotides at that position). In certain embodiments, the variant sequences include sequences differing from the known target sequence at two or more positions. Accordingly, in another embodiment, the putative target sequences included comprise the known target sequence for the endonuclease and all possible single and double variants of these (all possible double variants refers to the situation where all possible single variants are included in combination with every other possible single variant). In another embodiment, the putative target sequences included comprise the known target sequence for the endonuclease and all possible single, double and triple variants of these (all possible triple variants occurs where all possible single variants are included in combination with all possible double variants).

In one embodiment, the putative target sequences included comprise the known target sequence for the endonuclease and variants which differ from the known target sequence at a contiguous stretch of between 4 to 7 nucleotides. In one embodiment, a 4-7 stretch of nucleotides in the known target are modified to include all other possible 4-7 nucleotide combinations. In one embodiment, the putative target sequences include variants in which every 4-7 nucleotide stretch within the known target sequence is modified to include all other possible 4-7 nucleotide combinations.

In one embodiment where the substrate library comprises double stranded DNA substrates, the substrate library is tailored to a meganuclease (also known as a homing endonuclease) of the enzyme class EC 3.1.21. Examples of meganucleases include I-CreI, I-SceI and I-DmoI. The wild type version of the meganuclease I-CreI has been shown to recognise the sequence TGTTCTCAGGTACCTCAGCCAG (SEQ ID NO: 1). In one embodiment, a substrate library based on this known target sequence can be prepared. In one embodiment, the invention provides a substrate library wherein the putative target sequences present in the library as a whole includes SEQ ID NO: 1 and all possible single variants of SEQ ID NO: 1. In another embodiment, the invention provides a substrate library wherein the putative target sequences present in the library as a whole includes SEQ ID NO: 1 and all possible single and double variants of SEQ ID NO: 1. In another embodiment, the invention provides a substrate library wherein the putative target sequences present in the library as a whole includes SEQ ID NO: 1 and all possible single, double and triple variants of SEQ ID NO: 1.

The wild type version of the meganuclease I-SceI has been shown to recognise the sequence TAGGGATAACAGGGTAAT (SEQ ID NO: 2). In one embodiment, a substrate library based on this known target sequence can be prepared. In one embodiment, the invention provides a substrate library wherein the putative target sequences present in the library as a whole includes SEQ ID NO: 2 and all possible single variants of SEQ ID NO: x1. In another embodiment, the invention provides a substrate library wherein the putative target sequences present in the library as a whole includes SEQ ID NO: 2 and all possible single and double variants of SEQ ID NO: 2. In another embodiment, the invention provides a substrate library wherein the putative target sequences present in the library as a whole includes SEQ ID NO: 2 and all possible single, double and triple variants of SEQ ID NO: 2. The preparation of a substrate library based on this target sequence is described in Example 6.

In one embodiment where the substrate library comprises double stranded DNA substrates, the substrate library is tailored to a zinc finger nuclease. Whilst these nucleases are not naturally occurring, a number have been generated. Indeed, there are a number of publicly available systems for generating zinc finger nucleases including the Oligomerized Pool Engineering (OPEN), Context-Dependent Assembly (CoDA), and a bacterial one-hybrid (B1H) selection-based system The OPEN strategy has been used to generate zinc finger nucleases recognising particular sequences in endogenous human and tobacco genes (Maeder et al., Molecular Cell, 2008, 31(2): 294-301). In this study, a number of zinc finger nucleases were generated including a zinc finger nuclease capable of recognising the sequence CTACCCCGACCACATGAAGCAGCAC (SEQ ID NO:3). In one embodiment, a substrate library based on a target sequence of a zinc finger nuclease can be prepared. In one embodiment, the invention provides a substrate library wherein the putative target sequences present in the library as a whole includes SEQ ID NO: 3 and all possible single variants of SEQ ID NO: 3. In another embodiment, the invention provides a substrate library wherein the putative target sequences present in the library as a whole includes SEQ ID NO: 3 and all possible single and double variants of SEQ ID NO: 3. In another embodiment, the invention provides a substrate library wherein the putative target sequences present in the library as a whole includes SEQ ID NO: 3 and all possible single, double and triple variants of SEQ ID NO: 3.

In one embodiment where the substrate library comprises double stranded DNA substrates, the substrate library is tailored to a TALEN. Again, these are not naturally occurring, but a number have been generated and software and platforms exist to design and synthesise TALENs with particular specificities (e.g. TALEN targetter, E-TALEN, FLASH, Golden Gate). Reyon and colleagues used the FLASH system to produce TALENs targeting a number of human genes (Reyon et al., Nature Biotechnology, 2012, 30: 460-465). The TALEN targeting ERCC2 recognised the sequence TCCGGCCGGCGCCATGAAGTGAGAAGGGGGCTGGGGGTCGCGCTCGCTA (SEQ ID NO: 4). In one embodiment, a substrate library based on this known target sequence can be prepared. In one embodiment, the invention provides a substrate library wherein the putative target sequences present in the library as a whole includes SEQ ID NO: 4 and all possible single variants of SEQ ID NO: 4. In another embodiment, the invention provides a substrate library wherein the putative target sequences present in the library as a whole includes SEQ ID NO: 4 and all possible single and double variants of SEQ ID NO: 4. In another embodiment, the invention provides a substrate library wherein the putative target sequences present in the library as a whole includes SEQ ID NO: 4 and all possible single, double and triple variants of SEQ ID NO: 4.

In one embodiment, the substrate library is tailored to an RNA guided nuclease. RNA guided nucleases refer to nucleases that interact with a guide RNA (gRNA) and, in association with the gRNA, cleave a target region which may be double stranded or single stranded. gRNAs can be unimolecular (comprising a single RNA molecule) or modular comprising both a CRISPR RNA (crRNA) and a trans-activating crRNA (tracrRNA). gRNAs whether unimolecular or modular comprise a guide sequence that is complementary to the DNA sequence that is desired to be cleaved. In one embodiment, RNA guided nucleases include, but are not limited to naturally occurring class 2 CRISPR nucleases such as Cas9 or Cpf1, and variants of these which cut double stranded DNA. In one embodiment, the Class II CRISPR nuclease is naturally occurring. The target sequence of such nucleases will depend upon the nature of the class of nuclease and the gRNA in a manner that is well understood. For example, in general Cas9 nucleases recognise sequences in which a PAM (protospacer adjacent motif) sequence is 3′ of a protospacer sequence that is complementary to the guide sequence. Example 1 exemplifies a substrate library containing as putative target sequences, a 22 bp TCRα target region and all possible single, double and triple variants of this 22 bp sequence. The substrate library prepared in Example 1 was used in Example 5 to characterise cleavage of a wild type CRISPR Cas9 enzyme from S. pyogenes or a variant of this that exhibits the point mutation R691A that exhibits reduced off target activity, combined with a TCRα crRNA and a commercially available 67 bp tracrRNA that is modified to increase nuclease resistance. In another embodiment, RNA guided nucleases include, but are not limited to naturally occurring Cas9 enzymes of the type II-C subclass or variants thereof, or and Cas3 enzymes or variants of these which cut single stranded DNA.

In another embodiment, the substrate library is tailored to a known SNP that is known to be correlated with a particular disease indication. In this embodiment, the putative target sequences would include a sequence of 9-50 nucleotides in length including the SNP and surrounding wild type sequences, wherein in different putative target sequences the position of the SNP within the sequence is moved by a single nucleotide position. In addition to this, putative target sequences would include wild type sequences corresponding to all of the above sequences for all naturally occurring isoforms.

In one embodiment, a substrate library will contain over 1000 DNA substrates. In a more particular embodiment, a substrate library will contain over 10000 DNA substrates. In a more particular embodiment, a substrate library will contain over 60000 DNA substrates. In a more particular embodiment, a substrate library will contain over 100000 DNA substrates. In one embodiment of the substrate libraries described herein, the DNA substrates will be double stranded. In another embodiment of the substrate libraries described herein, the DNA substrates will be single stranded. The inventors have generated libraries of approximately 290000 substrates for use in the method of the invention.

In particular embodiments of the invention, each substrate in the substrate library is present in approximately the same copy number, in other words, the substrates are approximately equally abundant. Abundance of the substrates can be assessed by the method described in Example 3. In one embodiment, the abundance of at least 99% substrates in the library varies less than 5 fold, and in a more particular embodiment, less than 2 fold.

In addition to containing a putative target sequence, each substrate in the library also contains an identifier sequence. This is a DNA sequence that is uniquely present in combination with a particular target sequence such that it can act as a barcode. The exact sequence of the identifier sequence is not important and any sequence could be used as long as it can be linked back to a particular putative target sequence. There is one important restriction however, and that is that the sequence of the putative target sequence should not be the same as the sequence of the identifier sequence. In one embodiment, the sequence of each putative target sequence is not identical to the sequence of the related identifier sequence. In another embodiment, the sequence of an identifier sequence is not identical to the sequence of any putative target sequence present in the library. This ensures that the identifier sequence will remain intact when the putative target sequence is cleaved by an endonuclease.

In one embodiment, each unique identifier sequence present in the substrate library is the same length. In one embodiment, each unique identifier differs from every other unique identifier sequence by at least 1 nucleotide. In a more particular embodiment, each unique identifier differs from every other unique identifier sequence by at least 2 or at least 3 nucleotides. Having more than one distinct nucleotide minimising the likelihood of a sequencing error generating the sequence of another identifier sequence in the library.

In one embodiment, the unique identifiers do not include sequences that are internally complementary, or with homology to themselves, primers or other portions of the substrate.

In one embodiment, each of the putative target sequences in the substrate library is the same length as the other putative target sequences in the substrate library, and each of the identifier DNA sequences in the substrate library is the same length as the other identifier DNA sequences in the substrate library.

Each substrate in the library also contains a sequence that is complementary to a reverse PCR primer. This sequence is identical in each member of the library. The exact sequence is not important provided that the reverse PCR primer is capable of amplification of the substrate under appropriate conditions.

In certain embodiments where the DNA substrates are double stranded, each substrate in the library also contains a sequence that is complementary to a forward primer, where this sequence is located 5′ to the putative target sequence. This sequence is identical in each member of the library. As with the sequence complementary to the reverse primer, the exact sequence of the region complementary to a forward primer is not important provided that the forward PCR primer is capable of amplification of the substrate under appropriate conditions.

In certain embodiments, where the DNA substrates are double or single stranded, the DNA substrates has an affinity tag at its 5′ end. In certain embodiments where the DNA substrate is single stranded, the 5′ affinity tag chosen prevents ligation of the 5′ end to double stranded DNA, for example, by attaching to the DNA substrate via its 5′ hydroxyl.

The relative positioning of the sequence elements within each substrate is also important. It is important the sequences that are complementary to the forward and reverse primers are at the 5′ and 3′ termini respectively, such that they flank the putative target and identifier target sequences. As a result, PCR amplification using the forward and reverse primers would amplify both the putative target and identifier sequences.

In addition, it is important that the putative target sequence is 5′ (or upstream) of the identifier sequence. This is important for the use of the library in a method for identifying a target sequence as will be explained later. Where DNA substrates are double stranded, the skilled person will appreciate that the order of the sequence elements will only appear in one of the two strands. For the avoidance of doubt, a double stranded DNA substrate is a DNA substrate of the invention where the sequence elements are in the correct order in one of the two strands.

The various sequence elements (putative target sequence, identifier target sequence, sequence complementary to the reverse primer) may be separated from one another by DNA spacers. Typically, these are between 1-20 nucleotides in length. The precise sequence of any spacers is not important, although it will be appreciated that they should not have the same sequence as a putative target sequence or an identifier sequence. In one embodiment, the spacer sequences do not contain a known target sequence of an endonuclease. In one embodiment, the spacer between the identifier target sequence and the putative target sequence is between 1-20 nucleotides. In another embodiment, the spacer between the identifier target sequence and the putative target sequence is between 1-10 nucleotides. In a further embodiment, the spacer between the identifier target sequence and the putative target sequence is between 1-5 nucleotides. In a further embodiment, the spacer between the identifier target sequence and the putative target sequence is not more than 2 nucleotides.

The substrate library can be synthesised by conventional methods e.g. mutagenesis methods.

A commonly used method of generating degenerate oligos is to use mixed phosphoramidites (aka amidites, the building blocks of oligo synthesis) at desired positions in an oligo, e.g. using “N” to incorporate dA, dC, dG, and dT nucleotides, or “Y” for pyrimidines, “R” for purines. During automated chemical synthesis of oligos, the synthesizer consecutively adds dT, dA, dC, or dG in the case of “N” at a pre-set ratio (e.g. 25% each). This procedure does not always result in expected usage of each amidite because different amidites have different coupling efficiency, and the order of addition may also bias against amidites that are added later.

Using mixed bases as described above can result in limited control to achieve ratios of codons for specific amino acids. By using trimer amidites, which can be used for adding 3 nucleotides in each synthesis cycle, oligos encoding selected amino acids at pre-determined percentages can be created. However, this procedure is difficult to perform because trimer amidites are bulky and hard to couple to the elongating oligo; any moisture present during synthesis would have even more severe adverse effects than with regular amidites.

Another method for making library oligos is the “split-and-pool” method, which is particularly suitable for having diversified amino acids embedded in otherwise common sequences like the CDRs within antibody variable regions.

Additionally, DNA pools can be generated by error-prone PCR, or more specifically with overlapping PCR using degenerate primers.

Libraries of the type disclosed here could also be ordered from a commercial vendor such as Twist Bioscience.

In one embodiment where the substrate library comprises double stranded DNA substrates, the substrate library is obtained in a two step process. The first step comprises preparation of the putative target sequences. The putative target sequences may be derived from a known target sequence by a mutagenesis method or from a commercial vendor such as Twist Bioscience. Importantly, flanking the putative target sequence, the sequence must comprise sequences complementary to the primer sequences used method of identifying a DNA target sequence described herein and the additional sequence elements present in the library. The substrate library is generated by PCR amplification of the putative target sequence using 5′ and 3′ primers. Importantly, this means that the 3′ primer is not a single sequence but a plurality of sequences containing distinct identifier sequences together with a sequence complementary to the reverse primer. In embodiments where the substrate library contains a contains a sequence complementary to the forward primer, this is encoded by the 5′ primer used.

Accordingly, in one embodiment, the invention provides a method for preparing a substrate library comprising double stranded DNA substrates as defined herein comprising a step of PCR amplification of a plurality of putative target sequences flanked with a) a library forward primer and b) a sequence identical to a portion of a library reverse primer sequence, with said library forward primer and library reverse primer, wherein the library reverse primer is a heterogeneous mixture of DNA sequences containing distinct identifier sequences located 5′ of a sequence common to all sequences that is complementary to a reverse primer sequence, and wherein the number of distinct identifier sequences is in molar excess of the number of putative target sequences.

In one embodiment, the plurality of putative target sequences are all sequences of the same length.

The substrate library described herein may be used in a method for identifying a DNA target sequence of an endonuclease. Accordingly, in a second aspect, the invention provides a method for identifying a DNA target sequence of an endonuclease, comprising the following steps:
a) contacting a substrate library as defined herein with an endonuclease under suitable conditions to permit cleavage;
b) ligating the endonuclease treated library with a DNA sequence including a sequence complementary to a “cleavage” PCR primer;
c) PCR amplification of the cleaved substrate with cleavage and reverse PCR primers; and
d) sequencing of the amplified PCR product;

wherein a DNA target sequence in the cleaved product is identified via the sequence of the identifier sequence.

Whilst not essential, the selection of the endonuclease and library may be matched (i.e. so that the putative target sequences include any known target sequences of the endonuclease concerned and variants thereof). For example, where the nuclease used in the assay is I-CreI, a suitable substrate library would be a library including the sequence TGTTCTCAGGTACCTCAGCCAG (SEQ ID NO: 1) and variants thereof.

The method steps a) to d) enable identification of the putative target sequences that are cleaved by an endonuclease. Once identified, the putative target sequences are referred to as DNA target sequences.

The assay enables those sequences that are cleaved and hence the contained DNA target sequence to be identified as follows. First, the library is contacted by an endonuclease. Where a subset of the substrates contain DNA target sequences that are recognised by the endonuclease, these substrates will be cleaved. The library is then ligated to a DNA sequence complementary to a cleavage PCR primer. This ligation enables the cleaved substrates to be selectively amplified using the cleavage and reverse primers. The amplified DNA can then be sequenced to identify the DNA target sequence. In view of the fact that this sequence has been cleaved, this cannot be done directly, but the DNA target sequence can be indirectly identified by means of the identifier sequence which is unique to the DNA target sequence.

Step a) involves treatment of a substrate library with an endonuclease. The endonuclease may be an engineered nuclease such as a TALEN, or zinc finger nuclease. In one embodiment, the nuclease is a naturally occurring nuclease such as a meganuclease (homing endonuclease) or a naturally occurring RNA guided nuclease. In another embodiment, the endonuclease may be an organic compound nuclease, an enediyne, an antibiotic nuclease, dynemicin, neocarzinostatin, calicheamicin, esperamicin or bleomycin. In one embodiment, the nuclease is an engineered meganuclease or RNA guided nuclease that differs from a naturally occurring meganuclease by one or more residues.

It is important to note that the different substrates in the library do not need to be physically separated in order for the method to work. Steps (a), (b) and (c) may be conducted on the entire substrate library. Dilution of the PCR product of step (c) to a level where ˜60-70% of randomly formed droplets in an emulsion will contain precisely 1 DNA fragment, permits the sequencing of the individual fragments.

It follows that step (a) may be conducted as a one-pot reaction in which the entire substrate library is contacted with the endonuclease. Suitable conditions would be well understood by the skilled person and could be optimised for each endonuclease, but in summary step (a) takes place in solution at a suitable temperature, in a suitable buffer solution for a suitable time. Where the same buffer is used for steps (a) and (b) of the method, it is important that the buffer selected is suitable to permit the reactions in both of these steps to take place (e.g. broadly compatible with DNA resection and ligation).

In some embodiments where the substrate library comprises double stranded DNA substrates, endonuclease cleavage results in a blunt cleaved end. In other embodiments, endonuclease cleavage of double stranded DNA substrates results in an overhang or sticky end. In certain embodiments, in which endonuclease cleavage results in a sticky end, the method may comprises a step in which the sticky end is converted into a blunt end. This may be achieved by any appropriate method. Methods of blunting 5′ overhangs are known in the art. For example, a 5′ overhang may be blunted by filling in using a 5′ to 3′ DNA polymerase such as T4 polymerase or DNA polymerase I or functional fragments thereof (e.g. the large Klenow fragment of DNA polymerase I). For example, in example 7, a 5′ overhand is lunted by filling in with Klenow polymerase. Alternatively, a 5′ overhang may be blunted using a 5′ to 3′ exonuclease such as Mung Bean nuclease or a functional fragment thereof. Methods of blunting 3′ overhangs by filling in and or 3′ to 5′ exonuclease digestion are also well known.

In one embodiment, the reaction is quenched after step (a), after the (optional) step of generating a blunt end, or both to inactivate the enzymes. Any suitable method to inactivate the enzymes may be employed, but care should be taken to avoid the introduction of substances that would interfere with the subsequent steps of the method. In one embodiment, the reaction is quenched by heating to a temperature suitable to inactivate the enzymes but not to denature the DNA substrates, for example between 65-70° C. In another embodiment, necessary co-factors for the enzymes are removed, for example using a chelating agent, such as EDTA. In another embodiment, enzymes are physically removed, for example using a capture resin such as Ni-NTA-agarose or Streptavidin-agarose. In another embodiment, enzymes are destroyed through the use of promiscuous proteases such as Proteinase K.

Step (b) involves ligation in the presence of DNA sequence including a sequence complementary to a cleavage PCR primer. The DNA sequence including a sequence complementary to a cleavage PCR primer should be present in molar excess. In one embodiment, the DNA sequence including a sequence complementary to a cleavage PCR primer is present in a molar ratio of at least 3:1 with respect to the library DNA. In one embodiment, the DNA sequence including a sequence complementary to a cleavage PCR primer additionally contains a cleavage event identifier sequence (termed the well barcode oligonucleotide in the examples).

Any suitable DNA ligase (or functional fragments thereof) may be used in step (b). A number of DNA ligases (e.g. T4, T3, T7) are known in the art and many are commercially available. The type of ligase chosen for step (b) will depend upon the nature of the cleaved ends present following step (a) and upon the nature of the ends of the DNA sequence including a sequence complementary to a cleavage PCR primer. Where endonuclease cleavage generates blunt ends (or where overhangs are blunted before step b) and where the DNA sequence including a sequence complementary to a cleavage PCR primer also has a blunt 3′ end, a ligase suitable for ligating blunt ends may be chosen. Where endonuclease cleavage generates sticky ends, a ligase suitable for sticky ends may be selected. In embodiments where the substrate library is single stranded, the ligation step may employ a ligase suitable for ligating single stranded and double stranded DNA, for example circligase. In another embodiment where the substrate library is single stranded, the DNA sequence including a sequence complementary to a cleavage PCR primer can include degenerate sticky ends capable of hybridising to the cut single stranded DNA substrate. Where such a DNA sequence is used, a ligase capable of ligating sticky ends may be used.

As discussed above, in one embodiment, the DNA sequence including a sequence complementary to a cleavage PCR primer is blunt ended. In another embodiment, the DNA sequence including a sequence complementary to a cleavage PCR primer has a 3′ overhang. In a more particular embodiment, the overhang is between 1-10 nucleotides in length, more particularly, between 3-6 nucleotides in length, more particularly 4 nucleotides in length. Where the DNA sequence includes an overhang, ligation to the cleaved sequences is reduced, but the degree of noise (resulting from ligation to uncleaved sequences) is reduced significantly more, such that the signal to noise ratio improved.

In one embodiment, the ligase, and the DNA sequence including a sequence complementary to a cleavage PCR primer is added directly to the nuclease treated library of step (a). In an alternative embodiment, the ligase and the DNA sequence including a complementary to a cleavage PCR primer is added directly to the nuclease treated library following the steps of blunting a 5′ or 3′ overhang. Where co-factors required for the reaction are not present in the buffer, these may also be added at this stage.

In another embodiment, the ligase, and the DNA sequence including a sequence complementary to a cleavage PCR primer is added to the nuclease treated library of step (a) or to the blunted library following quenching of the reaction of step (a) (and/or where appropriate, the quenching of the blunting step). In these embodiments, the reaction of step (b) is conducted for a suitable time at a suitable temperature. Where co-factors required for the reaction are not present in the buffer, these may also be added at this stage. It is also necessary to ensure that the methods of quenching used previously do not interfere with the reaction of step (b).

In one embodiment, the DNA substrates in the substrate library include an affinity tag capable of being used to attach the substrate to a solid phase at their 5′ end. In one embodiment, the affinity tag is biotin, streptavidin or a histidine tag. Covalent capture tags such as thiol, disulphide, epoxide or aldehyde substrates can also be employed. Where the DNA sequence ligated onto the nuclease treated library is attached to an affinity tag capable of being used to attach the substrate to a solid phase, this may be used to separate the cleaved sequences and ligated substrates from the rest of the library. By melting apart or separating the individual strands of duplex DNA, only those which have been cleaved by an active endonuclease will be able to dissociate from the solid phase, leading to selective enrichment of the cleaved DNA fraction. In one embodiment, this melting process consists of raising the pH of the solution above 9, leading to dissociation of the DNA duplex. In other embodiment, this process may be achieved through heat or treatment with chaotropic agents such as guanidinium chloride, lithium perchlorate or urea. Whilst capture on a column or plate would be possible, capture on a bead is most compatible with the subsequent steps of the procedure. The skilled person would appreciate that capture would be effected on a coated bead (wherein the tag has affinity for the coating). Whilst not essential, this step increases the signal to noise ratio of the method.

Step c) involves PCR amplification of the cleaved substrate with cleavage and reverse PCR primers. In one embodiment, the product of step (b) is used directly in step (c), simply adding the required components of PCR (polymerase, nucleotides, primers, any necessary cofactors). In embodiments in which the uncleaved sequences are captured on a bead, either the DNA could be eluted from the beads and resuspended with the required components of PCR (including a suitable buffer) or the beads themselves could be resuspended in the required components of PCR including a suitable buffer. The requirements of PCR are well understood by the skilled person.

In one embodiment, the reverse primer includes an adaptor to facilitate subsequent next generation sequencing for a particular sequencing platform such as ION TORRENT NGS on e.g. a LIFE TECHNOLOGIES S5 SEQUENCER, a ROCHE 454A or 454B sequencing platform, an ILLUMINA SOLEXA sequencing platform, an APPLIED BIOSYSTEMS SOLID sequencing platform, a PACIFIC BIOSCIENCES'MRT sequencing platform, a POLLONATOR POLONY sequencing Platform, a HELICOS sequencing platform, a COMPLETE GENOMICS sequencing Platform, an INTELLIGENT BIOSYSTEMS sequencing platform, or for any other sequencing platform. In one particular embodiment, the reverse primer includes a reverse Illumina adaptor (e.g. i7).

In one embodiment, the reverse primer is attached to an affinity tag capable of being used to attach the substrate to a solid phase. In one embodiment, the affinity tag is biotin, streptavidin or a histidine tag. In another embodiment, the affinity tag is a covalent capture system and the tag is a thiol, disulphide, epoxide or aldehyde substrate.

Step (d) involves sequencing the amplified PCR product(s). Next generation sequencing techniques can be used here. Frequently, these require the products to be included on a solid phase. Where this is a requirement, the affinity tag on the reverse primer is used to capture the PCR products in a suitable format (e.g. on a plate or bead) for sequencing. It will be apparent that to the skilled reader that capture is accomplished by coating the plate or bead with the substance to which the tag has affinity. It will be also apparent to the skilled reader, that appropriate dilution prior to capture permits individual DNA fragments to be sequenced.

Whilst occasionally the sequence complementary to the cleavage primer will ligate to an uncleaved substrate, this is extremely rare and overwhelmingly only cleaved substrates and amplified by PCR. The sequence of the PCR products are identified in step (d). Whilst the DNA target sequence is not fully present in this sequence, this is unambiguously identified via the sequence of the identifier sequence. In those rare events where an uncleaved substrate is amplified, DNA sequencing would reveal that this includes the full putative target sequence. As a result, such “false positives” could be excluded from consideration (i.e. it would be appreciated that the contained putative target sequence is not a DNA target sequence).

In one embodiment, where the substrate library comprises double stranded DNA substrates and additionally contains a sequence complementary to a forward primer that is located 5′ to the putative target sequence, step c) further comprises PCR amplification of the uncleaved substrate with the forward and reverse PCR primers. Uncleaved substrates are the only substrates amplified with the forward primer. Additionally, uncleaved substrates can be further distinguished from cleaved sequences by the sequencing step (d). Uncleaved sequences will not contain the cleavage primer sequence and will contain intact putative target sequences and identifier sequences, whilst the cleaved sequences will contain the cleavage primer sequence, and an intact identifier sequence.

In one embodiment, where the substrate library comprises single stranded DNA substrates comprising a 5′ affinity tag that prevents ligation of double stranded DNA, step c) further comprises releasing uncut DNA substrates from the 5′ affinity tag, followed by the steps of ligating the uncut DNA substrates to a double stranded DNA sequence (containing in the 5′ to 3′ direction a sequence complementary to an uncut forward primer sequence) that is distinct from the cleavage primer sequence, and a step of PCR amplification using the uncut forward primer and reverse primer. Where the double stranded DNA sequence (containing in the 5′ to 3′ direction a sequence complementary to an uncut forward primer sequence has blunt ends, the ligation step may employ a ligase suitable for ligating single stranded and double stranded DNA, for example circligase. In another embodiment, the double stranded DNA sequence (containing in the 5′ to 3′ direction a sequence complementary to an uncut forward primer sequence has a sticky end which hybridises to a known sequence at the 5′ end of the uncut single stranded DNA substrate. Where this occurs, a ligase capable of ligating sticky ends may be used.

The conditions discussed in relation to step (b) and (c) above, are also suitable for the ligation and PCR steps used here.

The sequential removal of the cut and uncut substrates from the affinity tag permits separate amplification of the uncleaved and cleaved substrates. Additionally, uncleaved substrates can be further distinguished from cleaved sequences by the sequencing step (d). Uncleaved sequences will not contain the cleavage primer sequence and will contain intact putative target sequences and identifier sequences, whilst the cleaved sequences will contain the cleavage primer sequence, and an intact identifier sequence.

This embodiment of the method provides information on which sequences were cleaved and which were not. In the situation where the library contains multiple copies of each substrate, it may be the case that some copies of a putative target sequence were cleaved whilst others were not. Information on the proportion of sequences having a particular identifier that were cleaved gives information on which DNA target sequences are preferentially targeted by the nuclease.

In one embodiment, the DNA sequence including a sequence complementary to a cleavage PCR primer additionally contains a unique identifier sequence 5′ to the sequence complementary to the cleavage PCR primer. It will be evident that PCR amplification using the cleavage PCR primer and the reverse primer will give rise to a product containing two unique identifier sequences, one identifying the putative target sequence and the other identifying the ligation event. This controls for amplification bias and therefore permits a more accurate identification of the number of cleavage events.

In one embodiment, the uncut forward primer additionally contains a unique identifier sequence. It will be evident that PCR amplification using the uncut forward primer and the reverse primer will give rise to a product containing two unique identifier sequences, one identifying the putative target sequence and the other identifying the ligation event. This controls for amplification bias.

Similarly, repeating the method but changing the conditions of step (a) to reduce cleavage efficiency will also give information about which sequences are preferentially cleaved.

It will be appreciated that the sequencing step provides information not only as to whether the putative target sequence is a DNA target sequence, but also information as to the precise site of cleavage within the DNA target sequence. Accordingly, in one embodiment, the invention also provides a method for identifying the site of endonuclease cleavage in a DNA target sequence, comprising the following steps:
a) contacting a substrate library as defined herein with an endonuclease under suitable conditions to permit cleavage;
b) ligating the endonuclease treated library with a DNA sequence including a sequence complementary to a “cleavage” PCR primer;
c) PCR amplification of the cleaved substrate with cleavage and reverse PCR primers; and
d) sequencing of the amplified PCR product;

wherein a DNA target sequence in the cleaved product is identified via the sequence of the identifier sequence and wherein the site of endonuclease cleavage in a DNA target sequence is identified by sequencing of the amplified PCR product.

Importantly, this method can identify whether the exact site of cleavage is invariant or whether this can vary for any particular endonuclease.

The skilled reader would appreciate that in addition to identifying sequences that are preferentially cleaved at a single position, the method additionally generates information on the cleavage of related sequences which may be of relevance to off target binding and cleavage. It will be apparent that the information generated by this assay would enable the identification of enzymes having both activity for a particular sequence and additionally no activity for any related sequence that is additionally present in the genome. This is a particularly desirable trait in an enzyme is intended for gene therapy applications where cleavage at a single site is desired.

Example 7 confirms that the method of the invention is suitable to identify off target sequences. The top 25 DNA target sequences identified in this example include those identified those previously highlighted by other researchers using alternative methodology. Notably, this enabled the direct identification of true in vivo liabilities using a strictly in vitro assay, substantially simplifying the process of triaging enzymes possessing significant liabilities and tracking those liabilities in human cells.

In a further aspect, the method is conducted multiple times using same substrate library and variant endonucleases. For example, where the endonuclease is a meganuclease, the method is conducted using the wild type meganuclease and engineered forms of the meganuclease in which one or more residues are varied.

Collating information from variant endonucleases on the same substrates can inform which changes in the endonuclease modify target sequence specificity and can be used to guide further modification of the endonuclease to improve specificity for a particular DNA target sequence and/or reduce specificity for related sequences.

For example, frequently, the method will be conducted with a known endonuclease and a panel of variant endonucleases differing from that nuclease by one amino acid residue (single variants). In one embodiment, the panel of endonucleases includes all possible single amino acid variants (i.e. where the amino acid at each position is mutated to every other possible residue at that position). This permits the efficiency of cleavage by the variant endonucleases at particular substrate(s) to be compared. In this context, efficiency of cleavage refers to the percentage of cleaved sequences identified in the sequencing step. Where 100% of the sequences obtained in step d) of the method that have the relevant identifier are cleaved, this is considered 100% efficiency. Where 50% of the sequences with the relevant identifier are cleaved and 50% uncleaved, this is 50% efficiency. Similarly, where 30% of the sequences with the relevant identifier are cleaved and 70% uncleaved, this is 30% efficiency.

Accordingly, in another aspect, the invention provides a method for engineering endonucleases, comprising: a) conducting the method for identifying a DNA target sequence of a nuclease defined herein with a first endonuclease and at least two other endnucleases that differ from the first endonuclease by a single amino acid change at different positions within the endonuclease amino acid sequence using the same substrate library; b) comparing the efficiency of cleavage of each endonuclease tested in step a) at a particular substrate; c) identifying at least two amino acid changes at different positions that improve the efficiency of cleavage; and d) producing a variant endonuclease containing the at least two amino acid changes identified in step c).

The skilled reader would appreciate that this method can be extended to identify multiple amino acid substitutions that could improve the efficiency of cleavage of a substrate permitting the identification and production of variant endonucleases containing 3 or more amino acid substitutions.

Multiple substrates could be compared in this same way. Where one substrate is the desired target sequence and other sequences are related sequences present in the genome, variant sequences likely to improve cleavage efficiency at the desired target sequence whilst minimising cleavage efficiency at related genomic sequences could be identified. Accordingly, in another aspect, the invention provides a method for engineering endonucleases, comprising: a) conducting the method for identifying a DNA target sequence of a nuclease defined herein with a first endonuclease and at least two other endonucleases that differ from the first endonuclease by a single amino acid change at different positions within the endonuclease amino acid sequence using the same substrate library; b) comparing the efficiency of cleavage of each endonuclease tested in step a) at two separate substrates one of which is a desired target sequence and the other a related sequence present in the genome; c) identifying at least two amino acid changes at different positions that either improve the efficiency of cleavage at the desired target sequence or reduce the efficiency of cleavage at the related sequence present in the genome; and d) producing a variant endonuclease containing the at least two amino acid changes identified in step c).

The skilled reader would appreciate that this method can be extended to identify multiple amino acid substitutions that could improve the efficiency of cleavage of a desired target substrate and/or reduce the efficiency of cleavage at the related sequence present in the genome permitting the identification and production of variant endonucleases containing 3 or more amino acid substitutions.

Variant endonucleases produced according to the above methods also form an aspect of the invention. Variant endonucleases may have utility in the field of gene editing. Accordingly, in one embodiment, the invention provides a variant endonuclease for use in gene editing. In one embodiment, the invention provides a method for gene editing, which method comprises a step of transfecting DNA encoding a variant endonuclease into cells in vitro. In another embodiment, the invention provides DNA encoding a variant endonuclease for use in gene therapy. In another embodiment, the invention provides a method for gene therapy, which method comprises a step of administering a vector comprising DNA encoding a variant endonuclease into a patient in need thereof. In a further embodiment, the invention provides use of a vector comprising DNA encoding a variant endonuclease in the manufacture of a medicament for gene therapy.

### EXAMPLES

### Example 1: TCRα Substrate Library Preparation

A library of putative target sequences based upon a sequence present in the TCRα gene was ordered from Twist Biosciences. In particular, from an initial 22-bp target sequence including the 3′ PAM sequence GGN, in silico mutagenesis was used to generate all single, double and triple mutants therein, as well as all strings of 5 or more mutations in a row, amounting to 143,452 target sequences. Mutations included both the 3′ PAM sequence, as well as a 4 bp AAAA fragment added to the end of the pool as control bases. The following reaction was then conducted to convert the library of putative target sequences into a substrate library for use in a method of identification DNA target sequence of TCRα Cas9-Ribonucleotide:

| | |
|---|---|
| 1. | 28 μl of 100 uM biotin labelled |
| | library forward primer |
| 2. | 28 μl of100 uM biotin labelled |
| | library reverse primer containing |
| | heterogeneous identifier sequences |
| 3. | 4 μl library (10 ng/μl) |
| 4. | 80 μl 10 mM dNTPs |
| 5. | Water 3.02 ml |
| 6. | 800 μl 5X Phusion HF buffer |
| | (New England Biosciences B0518S) |
| 7. | 40 μl Phusion Hot Start II (2 U/μl) |

40 μl of the mixture is dispensed per well. The plate was then PCR amplified according to the following program:

| | |
|---|---|
| 1. | 98° C. 3 minutes |
| 2. | 98° C. 10 sec |
| 3. | 62° C. 30 sec |
| 4. | 72° C. 30 sec |
| | Repeat 2-4 12× |
| 5. | 4° C. hold |

The reactions from each PCR plate (c. 4 ml) were pooled. 0.1 volumes 3M NaOAc was added followed by 2.4 volumes absolute ethanol. The mixture was dispensed into microcentifuge tubes (1.4 ml/tube) which were incubated at −80° C. for at least one hour. The tubes were centrifuged at max rpm for 30 minutes and residual alcohol was aspirated (pellet not visible so aspiration is conducted on the side of the tube opposite to the one on the outside of the centrifuge). 200 μl 95% ethanol was added to each tube followed by vortexing. The tubes were then centrifuged at max rpm for 10 minutes and the supernatent was aspirated. The open tubes were placed in an incubator at 37° C. for at least 5 minutes followed by resuspending the pellets in a total of 500 μl water (split between the tubes). DNA was quantified by absorption at 260 nm using a Nanodrop 2000 Spectrophotometer.

### Example 2: Dynabead Preparation

Beads were prepared by placing 1 volume of streptavidin Dynabeads on a magnet and removing the storage buffer, followed by resuspending the beads in 1 volume of 1× wash buffer (5 mM Tris pH 7.5, 0.5 mM EDTA, 1M NaCl) followed by addition of 100 μM Random Hexamer (this may be sourced commercially, e.g., IDT DNA #51-01-18-27) oligonucleotides. The beads were then washed twice with one volume of 1× wash buffer and then once with 1 volume of 2× wash buffer prior to resuspension in 1 volume 2× wash buffer.

### Example 3: Substrate Library Characterisation

The substrate library prepared in example 1 was characterised to determine which oligonucleotides were present in the library and in what abundance. This characterisation provides a link between the putative target sequences to the identifier sequences present in the library.

50 μl reactions were prepared in multi-well plates as follows:

| | |
|---|---|
| 1. | 1000 ng substrate library |
| | prepared in example 1 |
| 2. | 5 μl 10X T4 DNA ligase buffer |
| 3. | 2.5 μl T4 DNA ligase |
| 4. | 2 μl 50 μM well barcode oligonucleotide |
| 5. | Water |

The reaction was incubated at 30° C. for 1.5 hour, then quenched at 65° C. for 20 minutes followed by holding at 4° C.

The ligated reaction product was purified by capture upon streptavidin beads (prepared as described in example 2).

50 μl beads were combined with 50 μl reaction. The mixture was washed 4 times with 100 μl 1× wash buffer, once with 50 μl 0.1× wash buffer, then twice with 50 μl 150 mM NaOH. The beads were resuspended in 50 μl 10 mM Tris pH 7.5. The beads were then used to prepare a 50 μl PCR reaction as follows:

| | |
|---|---|
| 1. | 5 μl beads |
| 2. | 10 μl 5 × Phusion HF buffer |
| | (New England Biosciences B0518S) |
| 3. | 0.25 μl primer containing plate barcode |
| 4. | 0.25 μl primer |
| 5. | 1 μl 10 mM dNTPs |
| 6. | 0.5 μl Phusion HS2 |
| 7. | 33 μl Water |

PCR amplification took place according to the following program:

| | |
|---|---|
| 1. | 98° C. 30 seconds |
| 2. | 98° C. 10 sec |
| 3. | 60° C. 5 sec |
| 4. | 72° C. 5 sec |
| | Repeat 2-4 9X |
| 5. | 12° C. hold |

Product was isolated using Pippin HT cleanup using the 3% cassette per manufacturer's instructions. Quantitative PCR was then performed and 50 μM products were loaded onto the Ion Chef using a whole 540 chip The 540 chip was then sequenced using a Life Technologies Ion S5 sequencer (A27212) according to manufacturer's instructions.

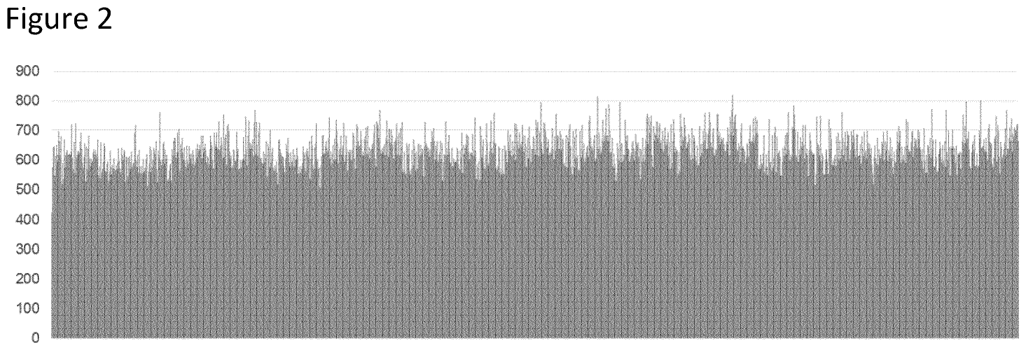

FIG. 2 shows the relative abundances of the DNA sequences present in the TCRα substrate library.

### Example 4: TCRα Cas9-Ribonucleoprotein Preparation

crRNA having the sequence GAGAAUCAAAAUCGGUGAAU (SEQ ID NO: 5) and a 67 bp universal tracrRNA (SEQ ID NO: 134 in U.S. Pat. No. 9,840,702; commercially available from IDT, catalogue number 1072532) were each reconstituted to 100 mM in water. Duplex gRNA was prepared by mixing equimolar amounts of crRNA and tracrRNA, heating to 95° C. for 3 minutes and allowed to cool to room temperature. Equimolar amounts of the duplex gRNA and either wild type CRISPR Cas9 enzyme from S. pyogenes or a R691A mutant enzyme were mixed to form active Cas9-ribonucleoprotein.

### Example 5: Identification DNA Target Sequence of TCRα Cas9-Ribonucleotide

10 μl cleavage reactions were prepared in multi-well plates as follows:

| | |
|---|---|
| 1. | 30 ng/μl substrate library prepared in example 1 |
| 2. | 1 mM MgCl₂ |
| 3. | 1 mg/ml bovine serum albumin |
| 4. | 10 mM Tris pH 7.5 |
| 5. | TCRα Cas9-ribonucleoprotein (variable concentrations - 8 μM, 4 μM, |
| | 2 μM and 0.4 μM) |

The cleavage reactions were incubated at 37° C. for 1 hour, then at 65° C. for 20 minutes. The following was then added to each well:

| | |
|---|---|
| 1. | 5 μl well barcode oligonucleotide |
| 2. | 5 μl ligation master mix (2.5 μl 10X T4 ligase buffer, 0.5 μl T4 |
| | ligase, 2 μl deionized water) |

The reactions were incubated at 30.5° C. for 1.5 hour, then at 65° C. for 20 minutes. Reactions using identical conditions (including the well barcode oligonucleotide) were pooled to ensure a total volume of at least 50 μl.

The above steps relate to steps (a) and (b) of the method for identifying a DNA target sequence described supra.

The library (both cleaved and uncleaved sequences) was purified by capture upon streptavidin beads (prepared as described in example 2).

50 μl beads were combined with 50 μl reaction. The mixture was washed 4 times with 100 μl 1× wash buffer, then once with 50 μl 0.1× wash buffer.

Cut DNA was then eluted by incubating beads with 50 μl 150 mM NaOH for 1 minute, then the supernatant placed in recipient wells containing 12 μl 1.25 M acetic acid and 6 μl 1M Tris pH 7.5, followed by a second incubation with an additional 50 μl 150 mM NaOH for 1 minute, which was then pooled with the first elution. Beads (containing uncut DNA) were then resuspended and stored in 50 μl 10 mM Tris pH 7.5.

Both cut and uncut DNA samples were used to prepare PCR reactions as follows:

PCR Reaction for Cut Samples

| | |
|---|---|
| 1. | 5 μl bead-purified supernatant containing cut product |
| 2. | 10 μl 5 × Phusion HF buffer (New England Biosciences B0518S) |
| 3. | 5 μl primer mix (5 μM primer complementary to the well barcode and |
| | containing the plate barcode + 5 μM primer complementary to the 3′ |
| | end of the oligonucleotide library) |
| 4. | 1 μl 10 mM dNTPs |
| 5. | 0.5 μl Phusion HS2 |
| 6. | 28.5 μl Water |

PCR amplification took place according to the following program:

| | |
|---|---|
| 1. | 98° C. 3 minutes |
| 2. | 98° C. 10 sec |
| 3. | 60° C. 30 sec |
| 4. | 72° C. 30 sec |
| | Repeat 2-4 11X |
| 5. | 4° C. hold |

PCR Reaction for Uncut Samples

| | |
|---|---|
| 1. | 5 μl beads |
| 2. | 10 μl 5 × Phusion HF buffer (New England Biosciences B0518S) |
| 3. | 0.25 μl primer containing plate barcode |
| 4. | 0.25 μl primer |
| 5. | 1 μl 10 mM dNTPs |
| 6. | 0.5 μl Phusion HS2 |
| 7. | 33 μl Water |

PCR amplification took place according to the following program:

| | |
|---|---|
| 6. | 98° C. 30 seconds |
| 7. | 98° C. 10 sec |
| 8. | 60° C. 5 sec |
| 9. | 72° C. 5 sec |
| | Repeat 2-4 9X |
| 10. | 12° C. hold |

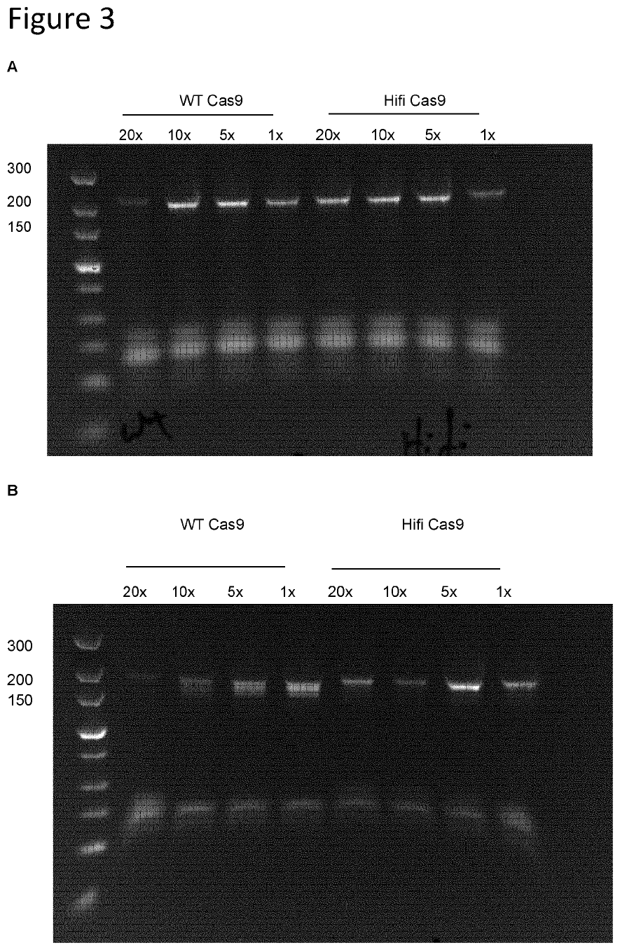

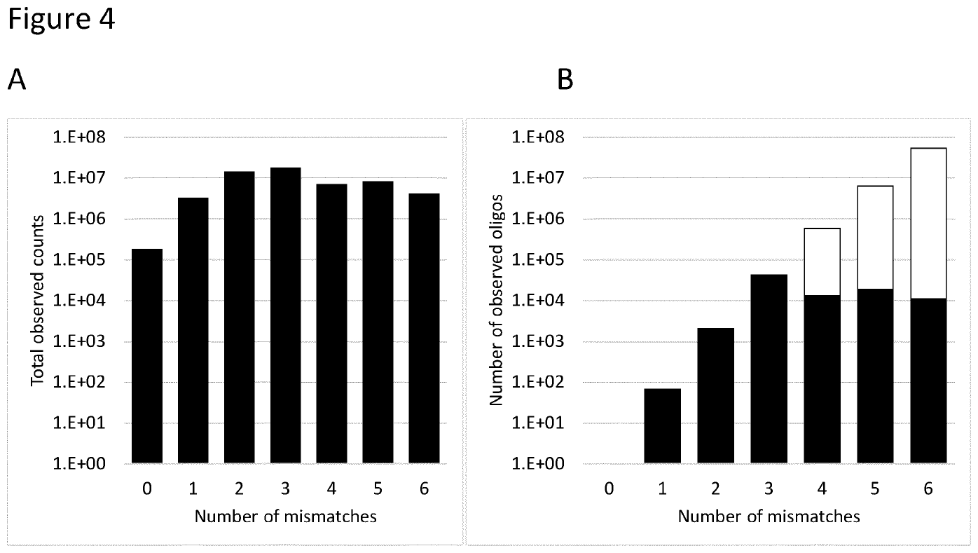

Samples of the uncut/cut PCR reactions were run on a 4% agarose gel. These are shown in FIG. 3. The gels confirm that the Cas-9 RNP cut and show that the wild type Cas9-RNP appears to have more nonspecific cleavage than the R691A mutant Cas9-RNP.

The cut/uncut DNA was isolated using Pippin HT cleanup using the 3% cassette per manufacturer's instructions. Quantitative PCR was then performed and 50 μM products were loaded onto the Ion Chef using a whole 540 chip. The 540 chip was then sequenced using a Life Technologies Ion S5 sequencer (A27212) according to manufacturer's instructions.

Pools were deconvoluted according to their well barcodes and analysed for overall cleavage frequencies. In each experiment, the raw abundance of each oligonucleotide was compared to the abundance of the true gRNA-targeted oligo, and evaluated for potential off-targets. These results are tabulated in Table 1.

TABLE 1

| [Cas9]/[DNA] | # > target, Cas9 | # > target, HiFi |
|---|---|---|
| 20 | 16,528 (30) | 16,556 (34) |
| 10 | 23,599 (27) | 18,603 (29) |
| 5 | 27,821 (27) | 18,227 (22) |
| 1 | 17,092 (30) | 7,559 (27) |
| 0.1 | 14,268 (40) | 9,889 (26) |
| 0.01 | 10,642 (84) | 10,208 (30) |
| 0.001 | 10,498 (48) | 10,642 (67) |
| 0.0001 | 9,713 (32) | 10,516 (58) |

In line with expectations, we observed decreasing numbers of off targets with decreasing enzyme:DNA stoichiometry. Surprisingly, we observed a ‘baseline specificity’ in each case, appearing at around 0.01-0.1 RNP:DNA ratio, where specificity ceased to improve with decreasing enzyme loading. This suggests an enzyme/gRNA-dependent irreducible background for the system.

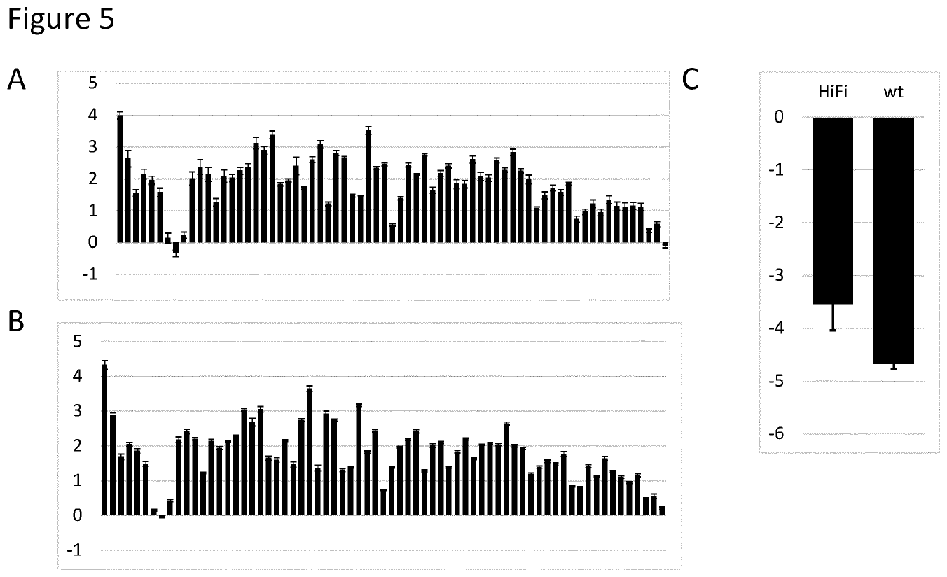

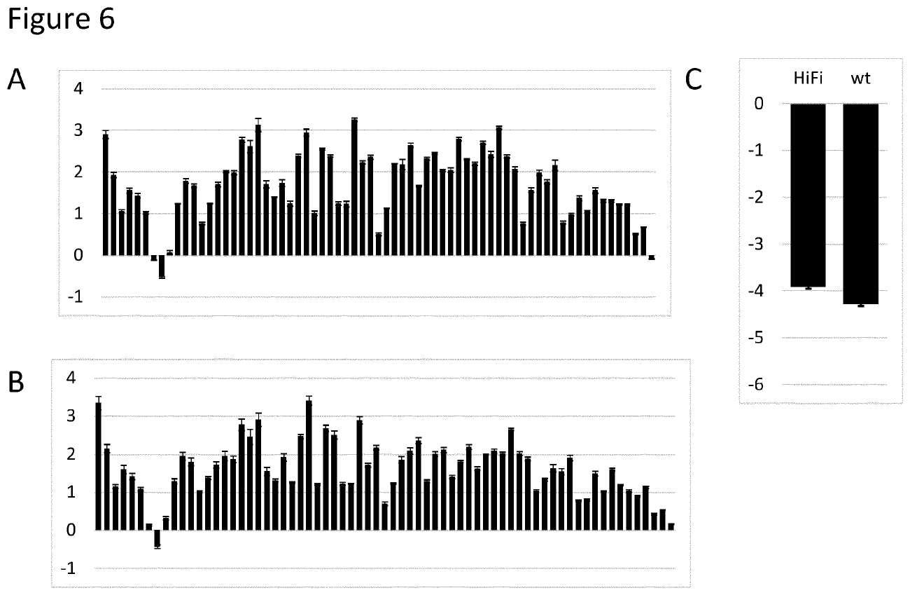

In addition, cut abundances were used to generate per-base pair binding energies using the BEESEM method reported by Zhao and Stormo (Nature Biotechnology 29, pages 480-483 (2011)). Samples were run as biological triplicates and then averaged to generate overall off-target binding penalties. These results for 1:1 RNP:DNA stoichiometry are shown in FIG. 5 and for 5:1 RNP:DNA stoichiometry are shown in FIG. 6. This analysis showed a clear correspondence in the per-base pair binding affinity for both HiFi and wild type spCas9, but we observed a statistically significant increase in the overall chemical potential term, indicating a general loss of activity. This is consistent with the previously reported mode of action of HiFi spCas9 derived through biophysical analysis of the HiFi spCas9 mutant (Nature Medicine 24, pages 1216-1224 (2018))

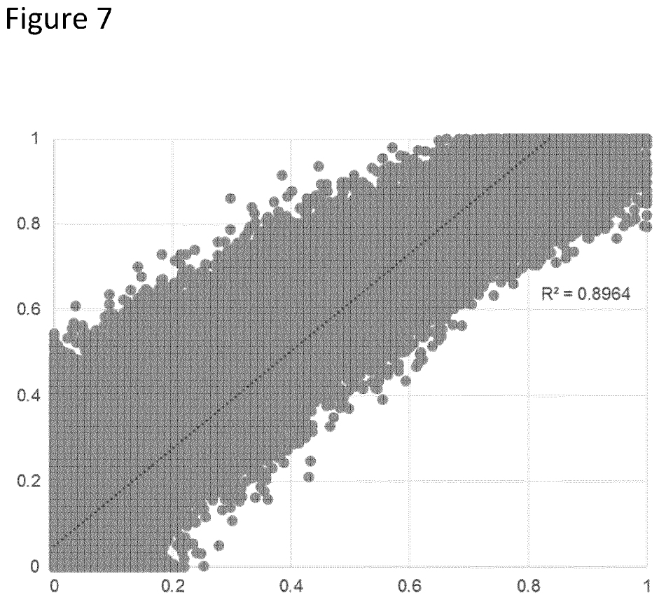

To test the reproducibility of individual oligo cleavage rates, these were computed using the BEESEM estimates and are shown in FIG. 7, demonstrating a high degree of reproducibility for the method.

### Example 6: I-SceI Substrate Library Preparation and Characterisation

I-SceI has the 18-base pair recognition sequence TAGGGATAACAGGGTAAT (SEQ ID NO: 2). A library containing the SEQ ID NO: 2 and all single, double and triple mutants of SEQ ID NO: 2 from the set [A,C,T,G] were enumerated, i.e. [AAGG . . . TAAT, TAGG . . . TGGA]. To this, all enumerations of a running window of size n=4 to 6, of all possible n-sized mutants i.e. [CCCC . . . TAAT, TAGG . . . CCCCCC], were included. The resulting library contained 59,914 members, each representing a putative target sequence. This library was ordered from Twist Biosciences.

An I-SceI substrate library was generated from the library of putative target sequences obtained from Twist Biosciences using the essentially the method described in Example 1 (with the minor difference that the concentration of the putative target sequences used in the PCR reaction was ˜9 ng/μl). Essentially, to each member of the pool a reference oligonucleotide with sequence CACGAGCGTAGCAGAGTATGTC (SEQ ID NO: 6) was prepended to the 5′ end of the putative target sequence, a “CG” spacer was placed between the putative target sequence and a unique identifier DNA sequence, and lastly a second reference oligonucleotide with sequence GAGCATGCTCTATCGTCTGATG (SEQ ID NO: 7) was appended to the 3′ end. An example pool member would have the constructed form: SEQ ID NO: 6-Putative Target Sequence-CG-Identifier DNA sequence-SEQ ID NO: 7.

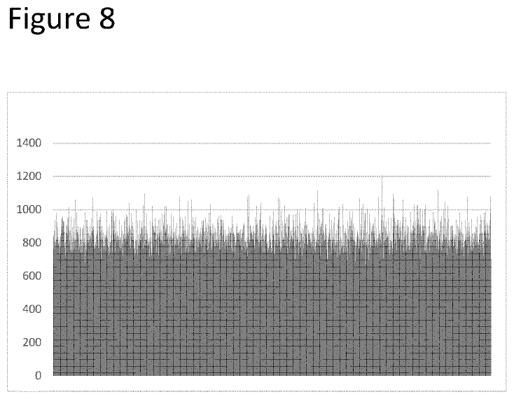

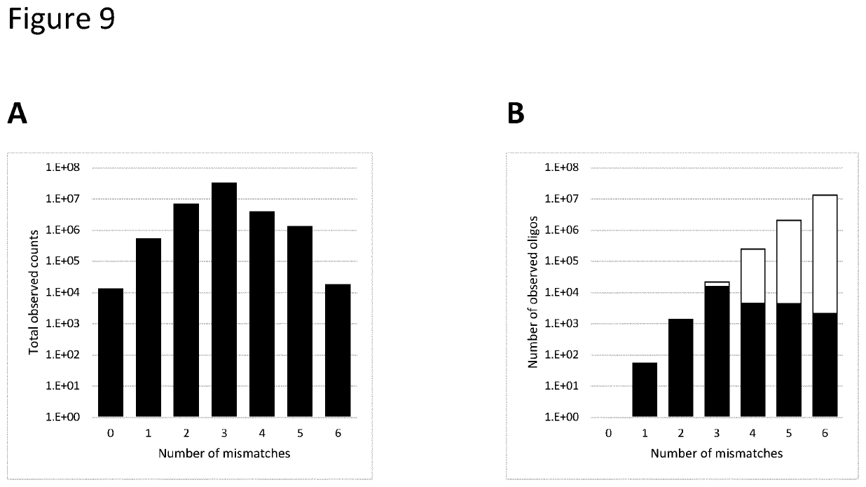

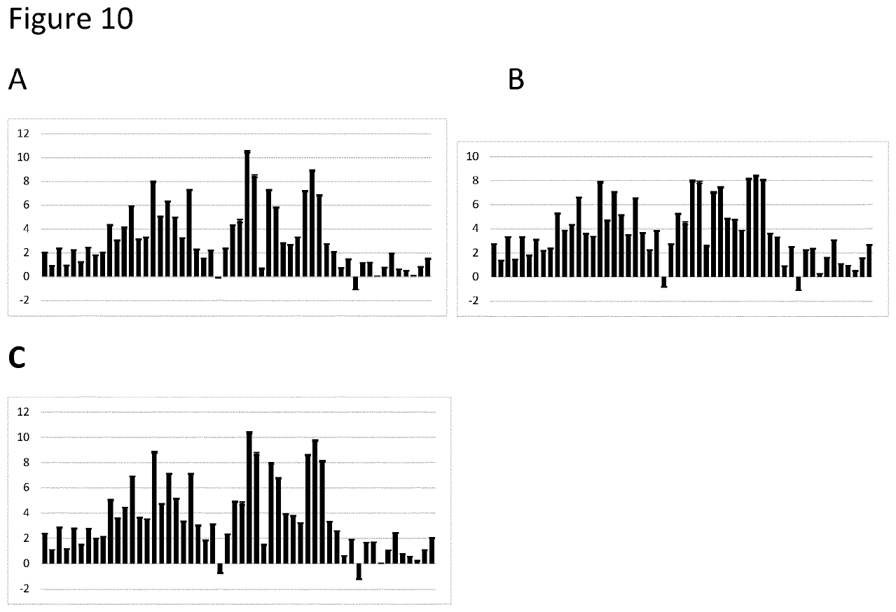

The I-SceI substrate library was characterised according to the method outlined in Example 3. FIG. 8 shows the relative abundances of the DNA sequences present in the I-SceI substrate library.

### Example 7: Identification DNA Target Sequence of I-SceI

Commercially available I-SceI was serially diluted as set out below:

| | |
|---|---|
| 1. | Neat |
| 2. | 1:10- 4 uL of neat into 36 uL H₂O |
| 3. | 1:100- 4 uL of 1:10 into 36 uL H₂O |
| 4. | 1:1,000- 4 uL of 1:100 into 36 uL H₂O |
| 5. | 1:10,000- 4 uL of 1:1000 into 36 uL H₂O |
| 6. | 1:100,000- 4 uL of 1:10000 into 36 uL H₂O |
| 7. | 1:1,000,000- 4 uL of 1:100000 into 36 uL H₂O |
| 8. | 1:10,000,000- 4 uL of 1:1000000 into 36 uL H₂O |

The I-SceI substrate library prepared as set out in Example 6 was diluted to ˜1000 ng/uL and used to prepare 10 μL cleavage reactions, set out below:

| | |
|---|---|
| 1. | 30 ng/μL I-SceI substrate library |
| 2. | 1 mM MgCl₂ |
| 3. | 1 mg/mL BSA |
| 4. | 10 mM Tris pH7.5 |
| 5. | 3 μl I-SceI (variable concentrations - dilutions prepared above) |

The plate was incubated at 37° C. for 1 hr followed by 65° C. for 20 min. This cleavage reaction relates to step (a) of the method for identifying a DNA target sequence described supra.

I-SceI cleavage results in overhanging single strands. These are “filled in” using Klenow polymerase by adding 5 uL Klenow mix (31.9 μl 10 mM dNTPs, 42.5 μL Klenow DNA Polymerase, 456.9 μL Deionized H₂O) to each cleavage reaction, sealing and incubating at room temperature for ˜30 mins before heat killing the enzyme at 65° C. for 20 mins.

The following was then added to each well:

| | |
|---|---|
| 1. | 5 μl well barcode oligonucleotide |
| 2. | 5 μl ligation master mix (2.5 μl 10X T4 ligase buffer, 0.5 μl T4 |
| | ligase, 2 μl deionized water) |

The reactions were incubated at 30.5° C. for 1.5 hour, then at 65° C. for 20 minutes before storing at 4° C. Reactions using identical conditions (including the well barcode oligonucleotide) were pooled to ensure a total volume of at least 50 μl. This relates to step (b) of the method for identifying a DNA target sequence described supra.

The library (both cleaved and uncleaved sequences) was purified by capture upon streptavidin beads (prepared as described in example 2).

50 μl beads were combined with 50 μl reaction. The mixture was washed 4 times with 100 μl 1× wash buffer, then once with 50 μl 0.1× wash buffer.

Cut DNA was then eluted by incubating beads with 50 μl 150 mM NaOH for 1 minute, then the supernatant placed in recipient wells containing 12 μl 1.25 M acetic acid and 6 μl 1M Tris pH 7.5, followed by a second incubation with an additional 50 μl 150 mM NaOH for 1 minute, which was then pooled with the first elution. Beads (containing uncut DNA) were then resuspended and stored in 50 μl 10 mM Tris pH 7.5.

Both cut and uncut DNA samples were used to prepare PCR reactions as follows:

PCR Reaction for Cut Samples

| | |
|---|---|
| 1. | 5 μl bead-purified supernatant containing cut product |
| 2. | 10 μl 5 × Phusion HF buffer (New England Biosciences B0518S) |
| 3. | 5 μl primer mix (5 μM primer complementary to the well barcode and |
| | containing the plate barcode + 5 μM primer complementary to the 3′ |
| | end of the oligonucleotide library) |
| 4. | 1 μl 10 mM dNTPs |
| 5. | 0.5 μl Phusion HS2 |
| 6. | 28.5 μl Water |

PCR amplification took place according to the following program:

| | |
|---|---|
| 1. | 98° C. 3 minutes |
| 2. | 98° C. 10 sec |
| 3. | 60° C. 30 sec |
| 4. | 72° C. 30 sec |
| | Repeat 2-4 15X |
| 5. | 4° C. hold |

PCR Reaction for Uncut Samples

| | |
|---|---|
| 1. | 5 μl beads |
| 2. | 10 μl 5 × Phusion HF buffer (New England Biosciences B0518S) |
| 3. | 0.25 μl primer containing plate barcode |
| 4. | 0.25 μl primer |
| 5. | 1 μl 10 mM dNTPs |
| 6. | 0.5 μl Phusion HS2 |
| 7. | 33 μl Water |

PCR amplification took place according to the following program:

| | |
|---|---|
| 1. | 98° C. 30 seconds |
| 2. | 98° C. 10 sec |
| 3. | 60° C. 5 sec |
| 4. | 72° C. 5 sec |
| | Repeat 2-4 9X |
| 5. | 12° C. hold |

Samples of the uncut/cut PCR reactions were run on a 4% agarose gel. Where a 180 bp fragment was not present in the cut reaction, an additional two cycles of PCR were performed.

The cut/uncut DNA was isolated using Pippin HT cleanup using the 3% cassette per manufacturer's instructions. Quantitative PCR was then performed and 50 μM products were loaded onto the Ion Chef using a whole 540 chip. The 540 chip was then sequenced using a Life Technologies Ion S5 sequencer (A27212) according to manufacturer's instructions.

Pools were deconvoluted according to their well barcodes and analysed for overall cleavage frequencies. In each experiment, the raw abundance of each oligonucleotide was compared to the abundance of the true gRNA-targeted oligo, and evaluated for potential off-targets. Using the derived scoring matrix from these data, the human reference genome was evaluated for potential off-target DNA sequences, and the top 25 putative off targets identified for each of various enzyme dilutions. Notably, this method was able to correctly all but one off targets detected using in vivo methods in the top 25 putative off-targets, greatly simplifying the workflow for identifying and evaluating potential genomic off-target sequences.

Table 2 identifies the top 25 DNA target sequences of I-SceI present in the human genome identified using this method. It is notable that this method identified all 5 of the sites observed in the previous work (Petek, Lisa M et al, “Frequent endonuclease cleavage at off-target locations in vivo.” Molecular Therapy 18.5 (2010): 983-986.) and 8 out of the 9 sites observed in a secondary study (Frock, Richard L, et al. “Genome-wide detection of DNA double-stranded breaks induced by engineered nucleases.” Nature biotechnology 33.2 (2015): 179-186.).

TABLE 2

| | | | | PWM | PWM | PWM | | |
|---|---|---|---|---|---|---|---|---|
| | | | | score, | score, | score, | Petek et | Frock et al, |
| RefLoc | Position | Strand | Targ Seq | Neat | 1: 10 | 1: 100 | al, 2010 | 2015 |
| NC_000001. | 181630062 | + | TAGGGATACC | 37 | 109 | 47 | Observed | Observed |
| 10 | | | AGGTCAAA | | | | | |
| | | | (SEQ ID NO: 8) | | | | | |
| NC_000013. | 40484114 | − | TAGGGATACC | −52 | −6 | −27 | | |
| 10 | | | AGGGTAGT | | | | | |
| | | | (SEQ ID NO: 9) | | | | | |
| NC_000017. | 56934496 | − | TAGGGATAAC | 53 | 42 | −36 | Observed | |
| 10 | | | AGGGCATA | | | | | |
| | | | (SEQ ID NO: | | | | | |
| | | | 10) | | | | | |
| NC_000005. | 13929909 | − | TAGGGATACC | −72 | −11 | −60 | | |
| 9 | | | AGGTTAAA | | | | | |
| | | | (SEQ ID NO: | | | | | |
| | | | 11) | | | | | |
| NC_000008. | 31472244 | − | TTGGGATAAC | −13 | −29 | −72 | | Observed |
| 10 | | | AGGGCAAT | | | | | |
| | | | (SEQ ID NO: | | | | | |
| | | | 12) | | | | | |
| NC_000005. | 84312457 | − | TAGGGATACC | −18 | 10 | −78 | | |
| 9 | | | AGGGCTGT | | | | | |
| | | | (SEQ ID NO: | | | | | |
| | | | 13) | | | | | |
| NC_000006. | 63726616 | − | TTGGGATACC | −52 | −12 | −83 | | |
| 11 | | | AGGGCATT | | | | | |
| | | | (SEQ ID NO: | | | | | |
| | | | 14) | | | | | |
| NC_000020. | 25775364 | − | CAGGGATAC | −37 | 0 | −85 | | |
| 10 | | | CAGGGCGGT | | | | | |
| | | | (SEQ ID NO: | | | | | |
| | | | 15) | | | | | |
| NC_000020. | 26041813 | + | CAGGGATAC | −37 | 0 | −85 | | |
| 10 | | | CAGGGCGGT | | | | | |
| | | | (SEQ ID NO: | | | | | |
| | | | 16) | | | | | |
| NC_000002. | 227922202 | − | CAGGGATAC | −54 | −25 | −103 | | |
| 11 | | | CAGGGCAAC | | | | | |
| | | | (SEQ ID NO: | | | | | |
| | | | 17) | | | | | |
| NC_000002. | 56645215 | + | CAGGGATAA | −54 | −50 | −120 | | |
| 11 | | | CAGGTCAAT | | | | | |
| | | | (SEQ ID NO: | | | | | |
| | | | 18) | | | | | |
| NC_000011. | 18703084 | − | TTGGGATAAC | −21 | −52 | −123 | Observed | Observed |
| 9 | | | AGGGCAAA | | | | | |
| | | | (SEQ ID NO: | | | | | |
| | | | 19) | | | | | |
| NC_000020. | 11479217 | + | TAGGGATACC | −111 | −95 | −154 | | Observed |
| 10 | | | AGGGTCAT | | | | | |
| | | | (SEQ ID NO: | | | | | |
| | | | 20) | | | | | |
| NC_000006. | 41996494 | + | TAGGGATAAC | −27 | −62 | −159 | | Observed |
| 11 | | | AGGGCTGT | | | | | |
| | | | (SEQ ID NO: | | | | | |
| | | | 21) | | | | | |
| NC_000008. | 135595511 | − | TAGGGATACC | −106 | −68 | −166 | | |
| 10 | | | AGGTCAAG | | | | | |
| | | | (SEQ ID NO: | | | | | |
| | | | 22) | | | | | |
| NC_000011. | 29529965 | + | TAGGGATACC | −139 | −92 | −167 | | Observed |
| 9 | | | AGGTTTAT | | | | | |
| | | | (SEQ ID NO: | | | | | |
| | | | 23) | | | | | |
| NC_000011. | 29530789 | + | TAGGGATACC | −139 | −92 | −167 | | |
| 9 | | | AGGTTTAT | | | | | |
| | | | (SEQ ID NO: | | | | | |
| | | | 24) | | | | | |
| NC_000009. | 24846362 | + | TAGGGATAAC | −85 | −87 | −169 | Observed | Observed |
| 11 | | | AGGTTGAA | | | | | |
| | | | (SEQ ID NO: | | | | | |
| | | | 25) | | | | | |
| NC_000015. | 64992978 | + | CAGGGATAA | −62 | −73 | −171 | | |
| 9 | | | CAGGTCAAA | | | | | |
| | | | (SEQ ID NO: | | | | | |
| | | | 26) | | | | | |
| NC_000016. | 16950402 | − | CAGGGATAC | −146 | −120 | −192 | Observed | |
| 9 | | | CAGGGTGGT | | | | | |
| | | | (SEQ ID NO: | | | | | |
| | | | 27) | | | | | |
| NC_000004. | 150238750 | − | TAGGGATGC | −104 | −91 | −193 | | |
| 11 | | | CAGGGCAGA | | | | | |
| | | | (SEQ ID NO: | | | | | |
| | | | 28) | | | | | |
| NC_000002. | 119931318 | + | TAGGGATGC | −156 | −137 | −220 | | Observed |
| 11 | | | CAGGGTGAA | | | | | |
| | | | (SEQ ID NO: | | | | | |
| | | | 29) | | | | | |
| NC_000003. | 110688942 | + | CAGGGATGC | −133 | −123 | −222 | | |
| 11 | | | CAGGGCAAA | | | | | |
| | | | (SEQ ID NO: | | | | | |
| | | | 30) | | | | | |
| NC_000015. | 45440233 | + | CAGGGATGC | −133 | −123 | −222 | | |
| 9 | | | CAGGGCAAA | | | | | |
| | | | (SEQ ID NO: | | | | | |
| | | | 31) | | | | | |
| NC_000005. | 122480727 | + | TAGGGATACC | −223 | −150 | −242 | | |
| 9 | | | ATGGCAAA | | | | | |
| | | | (SEQ ID NO: | | | | | |
| | | | 32) | | | | | |

1. A substrate library, comprising a plurality of DNA substrates, wherein each of the DNA substrates within the library contains a putative target sequence that is 5′ of an identifier DNA sequence capable of uniquely identifying the putative target sequence, which is 5′ of a sequence that is identical to a reverse PCR primer, wherein the DNA substrates within the library differ from one another only by the putative target sequences and the identifier DNA sequences, wherein each of the DNA substrates has an affinity tag at its 5′ end, and wherein the affinity tag is capable of being used to attach the DNA substrate to a solid phase.

2. A substrate library according to claim 1, wherein the DNA substrates are double stranded DNA substrates.

3. A substrate library according to claim 2, wherein each of the DNA substrates within the library additionally contains a sequence complementary to a forward primer, and wherein this sequence is located 5′ to the putative target sequence.

4. A substrate library according to claim 1, wherein the DNA substrates are single stranded substrates.

5. A substrate library according to claim 1, wherein the putative target sequence is not identical to the identifier sequence.

6. A substrate library according to claim 1, wherein each of the putative target sequences in the substrate library is the same length as the other putative target sequences in the substrate library, and wherein each of the identifier DNA sequences in the substrate library is the same length as the other identifier DNA sequences in the substrate library.

7. A substrate library according to claim 1, wherein the putative target sequences present in the library as a whole includes a characterised target sequence of an endonuclease and all possible single variants of this characterised target sequence.

8. A substrate library according to claim 7, wherein the endonuclease is an RNA guided nuclease, a meganuclease, a TALEN or a zinc finger nuclease.

9. (canceled)

10. A method for preparing a substrate library as defined in claim 2, comprising a step of PCR amplification of a plurality of putative target sequences flanked with a) a sequence complementary to a library forward primer and b) a sequence identical to a portion of a library reverse primer, with said library forward primer and library reverse primer, wherein the library reverse primer is a heterogeneous mixture of DNA sequences containing distinct identifier sequences located 5′ of a sequence common to all sequences that is complementary to the library reverse primer, and wherein the number of the distinct identifier sequences is in molar excess of the number of the putative target sequences.

11. A method for identifying a DNA target sequence of an endonuclease, comprising the following steps:
a) contacting a substrate library comprising a plurality of DNA substrates with the endonuclease under suitable conditions to permit cleavage, wherein each of the DNA substrates within the substrate library contains a putative target sequence that is 5′ of an identifier DNA sequence capable of uniquely identifying the putative target sequence, which is 5′ of a sequence that is identical to a reverse PCR primer, and wherein the DNA substrates within the substrate library differ from one another only by the putative target sequences and the identifier DNA sequences;
b) ligating the endonuclease contacted library from step a) with a DNA sequence including a sequence complementary to a cleavage PCR primer;
c) PCR amplification of a cleaved substrate in the ligated library from step b) with the cleavage primer and the reverse PCR primer to generate an amplified PCR product; and
d) sequencing of the amplified PCR product;
wherein the DNA target sequence of the endonuclease is identified via the identifier DNA sequence in the sequenced amplified PCR product from step d).

12. A method according to claim 11, wherein each of the DNA substrates within the library additionally contains a sequence complementary to a forward primer, wherein this sequence is located 5′ to the putative target sequence, and wherein step c) further comprises PCR amplification of a uncleaved substrate in the ligated library from step b) with the forward primer and the reverse PCR primer.

13. A method according to claim 11, wherein each of the DNA substrates has an affinity tag at its 5′ end, and wherein the affinity tag is used to attach the ligated library of step (b) to the solid phase, followed by a step of eluting the cleaved substrate.

14. A method according to claim 13, wherein the DNA substrates are single stranded substrates, and wherein, following the step of eluting the cleaved substrate and before step (d), there are the following steps:
i) cleavage of the affinity tag and elution of an uncleaved substrate;
ii) ligation of the uncleaved substrate to a double stranded DNA sequence containing in the 5′ to 3′ direction a sequence complementary to an uncut forward primer sequence; and
iii) PCR amplification of the cleaved substrate with the uncut forward primer and the reverse PCR primer.

15. A method according to claim 11, wherein the endonuclease is selected from the group consisting of an RNA guided nuclease, a meganuclease, a TALEN and a zinc finger nuclease.

16. A method according to claim 15, wherein the endonuclease is a naturally occurring or engineered meganuclease.

17. A method for engineering endonucleases, comprising:
i) conducting the method of claim 11 with a first endonuclease and at least two other endonucleases that differ from the first endonuclease by a single amino acid change at different positions using the same substrate library;
ii) comparing efficiency of cleavage of each of the endonucleases tested in step i) at a particular substrate;
iii) identifying at least two amino acid changes at different positions that improve the efficiency of cleavage; and
iv) producing a variant endonuclease containing the at least two amino acid changes identified in step iii).

18. A method according claim 17, wherein each of the endonucleases is selected from the group consisting of an RNA guided nuclease, a meganuclease, a TALEN and a zinc finger nuclease.

19. A variant endonuclease obtained by the method of claim 17.

20. A variant endonuclease according to claim 19 for use in gene editing.

21. A substrate library according to claim 1, wherein the affinity tag is biotin.


SEQUENCE LISTING (32 sequences)

SEQ ID NO: 1 - DNA, 22 bp, Artificial
  DNA recognition sequence of wild type I-CreI
tgttctcaggtacctcagccag

SEQ ID NO: 2 - DNA, 18 bp, Artificial
  DNA recognition sequence of wild type I-SceI
tagggataacagggtaat

SEQ ID NO: 3 - DNA, 25 bp, Artificial
  DNA recognition sequence of engineered Zn finger nuclease prepared by Maeder et al., Molecular Cell, 2008, 31(2) 294-301
ctaccccgaccacatgaagcagcac

SEQ ID NO: 4 - DNA, 49 bp, Artificial
  DNA recognition sequence of TALEN targetting ERCC2 prepared by Reyon et al., Nature Biotechnology, 2012, 30 460-465
tccggccggcgccatgaagtgagaagggggctgggggtcgcgctcgcta

SEQ ID NO: 5 - RNA, 20 nt, Artificial
  crRNA sequence targetting TCRa gene
gagaaucaaaaucggugaau

SEQ ID NO: 6 - DNA, 22 bp, Artificial
  Forward DNA primer for I-SceI substrate library production
cacgagcgtagcagagtatgtc

SEQ ID NO: 7 - DNA, 22 bp, Artificial
  Reverse DNA primer for I-SceI substrate library production
gagcatgctctatcgtctgatg

SEQ ID NO: 8 - DNA, 18 bp, Artificial
  off target DNA sequence of I-SceI
tagggataccaggtcaaa

SEQ ID NO: 9 - DNA, 18 bp, Artificial
  off target DNA sequence of I-SceI
tagggataccagggtagt

SEQ ID NO: 10 - DNA, 18 bp, Artificial
  off target DNA sequence of I-SceI
tagggataacagggcata

SEQ ID NO: 11 - DNA, 18 bp, Artificial
  off target DNA sequence of I-SceI
tagggataccaggttaaa

SEQ ID NO: 12 - DNA, 18 bp, Artificial
  off target DNA sequence of I-SceI
ttgggataacagggcaat

SEQ ID NO: 13 - DNA, 18 bp, Artificial
  off target DNA sequence of I-SceI
tagggataccagggctgt

SEQ ID NO: 14 - DNA, 18 bp, Artificial
  off target DNA sequence of I-SceI
ttgggataccagggcatt

SEQ ID NO: 15 - DNA, 18 bp, Artificial
  off target DNA sequence of I-SceI
cagggataccagggcggt

SEQ ID NO: 16 - DNA, 18 bp, Artificial
  off target DNA sequence of I-SceI
cagggataccagggcggt

SEQ ID NO: 17 - DNA, 18 bp, Artificial
  off target DNA sequence of I-SceI
cagggataccagggcaac

SEQ ID NO: 18 - DNA, 18 bp, Artificial
  off target DNA sequence of I-SceI
cagggataacaggtcaat

SEQ ID NO: 19 - DNA, 18 bp, Artificial
  off target DNA sequence of I-SceI
ttgggataacagggcaaa

SEQ ID NO: 20 - DNA, 18 bp, Artificial
  off target DNA sequence of I-SceI
tagggataccagggtcat

SEQ ID NO: 21 - DNA, 18 bp, Artificial
  off target DNA sequence of I-SceI
tagggataacagggctgt

SEQ ID NO: 22 - DNA, 18 bp, Artificial
  off target DNA sequence of I-SceI
tagggataccaggtcaag

SEQ ID NO: 23 - DNA, 18 bp, Artificial
  off target DNA sequence of I-SceI
tagggataccaggtttat

SEQ ID NO: 24 - DNA, 18 bp, Artificial
  off target DNA sequence of I-SceI
tagggataccaggtttat

SEQ ID NO: 25 - DNA, 18 bp, Artificial
  off target DNA sequence of I-SceI
tagggataacaggttgaa

SEQ ID NO: 26 - DNA, 18 bp, Artificial
  off target DNA sequence of I-SceI
cagggataacaggtcaaa

SEQ ID NO: 27 - DNA, 18 bp, Artificial
  off target DNA sequence of I-SceI
cagggataccagggtggt

SEQ ID NO: 28 - DNA, 18 bp, Artificial
  off target DNA sequence of I-SceI
tagggatgccagggcaga

SEQ ID NO: 29 - DNA, 18 bp, Artificial
  off target DNA sequence of I-SceI
tagggatgccagggtgaa

SEQ ID NO: 30 - DNA, 18 bp, Artificial
  off target DNA sequence of I-SceI
cagggatgccagggcaaa

SEQ ID NO: 31 - DNA, 18 bp, Artificial
  off target DNA sequence of I-SceI
cagggatgccagggcaaa

SEQ ID NO: 32 - DNA, 18 bp, Artificial
  off target DNA sequence of I-SceI
tagggataccatggcaaa